(19) United States
(12) Patent Application Publication
(10) Pub. No.: US 2026/0296308 A1
(43) Pub. Date: Oct. 1, 2026

(54) VEHICLE AND METHOD OF CONTROLLING VEHICLE

(71) Applicants: Hyundai Motor Company, Seoul (KR); Kia Corporation, Seoul (KR)

(72) Inventors: Ki Chang Kim, Hwaseong (KR); Dong Chul Park, Hwaseong (KR)

(21) Appl. No.: 19/310,751

(22) Filed: Aug. 26, 2025

(30) Foreign Application Priority Data
Mar. 28, 2025 (KR) 10-2025-0040163

(51) Int. Cl. B60Q 9/00; A61M 21/00; A61M 21/02; G07C 5/02

(52) U.S. Cl. CPC B60Q 9/00; A61M 21/02; G07C 5/02; A61M 2021/0022; A61M 2021/0027; A61M 2205/3303; A61M 2230/06; A61M 2230/40; A61M 2230/63

(57) ABSTRACT

According to embodiments, there is provided a vehicle comprising a sensor unit, one or more processors, and a memory configured to store one or more programs executed by the one or more processors, wherein the sensor unit detects traveling data of the vehicle and biometric information of a vehicle passenger, and the processor analyzes correlation between regenerative braking information of the traveling data of the vehicle, acceleration information generated during regenerative braking, and the biometric information, analyzes a risk of motion sickness of the passenger based on the correlation, and generates and outputs a motion sickness reduction feedback signal according to the risk of motion sickness. The motion sickness reduction feedback signal may comprise at least one of a sound signal and a vibration signal.

### CROSS-REFERENCE TO RELATED APPLICATION

This application claims, under 35 U.S.C. § 119(a), the benefit of Korean Patent Application No. 10-2025-0040163, filed on Mar. 28, 2025, in the Korean Intellectual Property Office, the entire contents of which are incorporated herein by reference.

### BACKGROUND

### (a) Technical Field

The present disclosure relates to Embodiments relate to a vehicle and a method of controlling a vehicle.

### (b) Description of the Related Art

With interest in eco-friendly vehicles increasing, the supply of electric vehicles is expanding, and various technologies are being developed to maximize the energy efficiency of electric vehicles. Unlike internal combustion engine vehicles, electric vehicles are driven by a motor and can perform regenerative braking by converting a driving motor into a generator when decelerating while traveling.

Regenerative braking is a method of converting kinetic energy of a vehicle into electric energy when decelerating and storing the electric energy in a battery. Regenerative braking provides a braking force that can decelerate or completely stop the vehicle without using a conventional friction brake. Accordingly, it is possible to reduce unnecessary energy loss and increase the energy efficiency of the vehicle by reusing the energy stored in the battery when accelerating.

However, the characteristics/braking feel of regenerative braking differs from conventional braking (e.g., the braking performed in conventional internal combustion engine vehicles). This difference can cause some passengers to experience motion sickness. In particular, when regenerative braking is strongly applied, when a deceleration pattern of a vehicle is not consistent, or when rapid acceleration and deceleration are repeated, passengers are more likely to feel motion sickness due to a mismatch between a vestibular organ and visual information. Such a phenomenon is especially noticeable for passengers sitting in the rear seats of a vehicle, since the perception of acceleration and deceleration is greater in the rear seats than in the front seats.

### SUMMARY

Various aspects of the disclosure provide a vehicle for controlling a vehicle in which a customized feedback system capable of preventing and alleviating motion sickness that may occur due to regenerative braking characteristics of an electric vehicle can be provided so that the riding comfort of passengers is improved and the energy efficiency of a vehicle is maximized, and a method of controlling the same.

According to embodiments, there is provided a vehicle comprising a sensor unit, one or more processors, and a memory configured to store one or more programs executed by the one or more processors, wherein the sensor unit detects traveling data of a vehicle and biometric information of a passenger of the vehicle, and the processor analyzes correlation between regenerative braking information of the traveling data of the vehicle, acceleration information generated during regenerative braking, and the biometric information, analyzes a risk of motion sickness of the passenger based on the correlation, and generates and outputs a motion sickness reduction feedback signal according to the risk of motion sickness.

The motion sickness reduction feedback signal my comprise at least one of a sound signal and a vibration signal.

The biometric information may comprise at least one of heart rate information, respiration information, skin stimulation information, and movement information of the passenger.

The regenerative braking information may comprise regenerative braking stage information.

The processor may comprise a motion sickness prediction model that analyzes the risk of motion sickness by analyzing the correlation between the regenerative braking information, the acceleration information, and the biometric information.

The motion sickness prediction model may learn a change in the biometric information according to a regenerative braking stage included in the regenerative braking information and the acceleration information.

The motion sickness prediction model may predict the risk of motion sickness based on the change in the biometric information.

The processor may generate and output a motion sickness-reduction feedback signal for each step based on the risk of motion sickness.

The processor may generate and output a sound signal for stabilizing a heart rate based on an acceleration generated during the regenerative braking being within a first threshold range.

The processor may generate and output a sound signal for stabilizing a respiration pattern based on an acceleration generated during the regenerative braking being within a second threshold range.

The processor may adjust an intensity and pattern of the vibration signal according to the risk of motion sickness.

According to embodiments, there is provided a method of controlling a vehicle which is performed by a computing device comprising a sensor unit, one or more processors, and a memory configured to store one or more programs executed by the one or more processors and which comprises detecting, by the sensor unit, traveling data of a vehicle and biometric information of a passenger of the vehicle, analyzing, by the processor, correlation between regenerative braking information of the traveling data of the vehicle, acceleration information generated during regenerative braking, and the biometric information, analyzing, by the processor, a risk of motion sickness of the passenger based on the correlation, and generating and outputting, by the processor, a motion sickness reduction feedback signal according to the risk of motion sickness.

The motion sickness reduction feedback signal may comprise at least one of a sound signal and a vibration signal.

The biometric information may comprise at least one of heart rate information, respiration information, skin stimulation information, and movement information of the passenger.

The regenerative braking information may comprise regenerative braking stage information.

The processor may comprise a motion sickness prediction model that analyzes the risk of motion sickness by analyzing the correlation between the regenerative braking information, the acceleration information, and the biometric information.

The analyzing of the correlation may comprise learning, by the motion sickness prediction model, a change in the biometric information according to a regenerative braking stage included in the regenerative braking information and the acceleration information.

The analyzing of the risk of motion sickness may comprise predicting, by the motion sickness prediction model, the risk of motion sickness based on the change in the biometric information.

The generating and outputting of the feedback signal may comprise generating and outputting a motion sickness-reduction feedback signal for each step based on the risk of motion sickness.

The generating and outputting of the feedback signal may comprise generating and outputting a sound signal for stabilizing a heart rate based on an acceleration generated during the regenerative braking being within a first threshold range.

The generating and outputting of the feedback signal may comprise generating and outputting a sound signal for stabilizing a respiration pattern based on an acceleration generated during the regenerative braking being within a second threshold range.

The generating and outputting of the feedback signal may comprise adjusting an intensity and pattern of the vibration signal according to the risk of motion sickness.

### BRIEF DESCRIPTION OF THE DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1 illustrates an exemplary view of a vehicle configured to communicate with another device to transmit and receive data, according to an exemplary embodiment of the present disclosure.

FIG. 2 illustrates a view of a module that constitutes a vehicle, according to an exemplary embodiment of the present disclosure.

FIG. 3 illustrates a system configured to enable a user to view virtual environment driving content, according to an exemplary embodiment of the present disclosure.

FIG. 4 illustrates a view a seat comprising a vibrator, according to an exemplary embodiment of the present disclosure.

FIG. 5 illustrates a schematic system for operating a vehicle, according to an exemplary embodiment of the present disclosure.

FIG. 6 illustrates a view of a vehicle configured to perform regenerative braking, according to an exemplary embodiment of the preset disclosure.

FIG. 7 illustrates a flowchart of a method for controlling a vehicle, according to an exemplary embodiment of the present disclosure.

### DETAILED DESCRIPTION

Hereinafter, the exemplary embodiment of the present disclosure will be described in detail. This exemplary embodiment is implemented based on the technical solution of the present disclosure, and shows a specific implementation method and a specific operation process, but the protection scope of the present disclosure is not limited to the exemplary embodiment below.

The following Detailed Description is merely provided by way of example and not of limitation. Furthermore, there is no intention to be bound by any expressed or implied theory presented in the preceding background or in the following Detailed Description.

Reference will now be made in detail to various exemplary embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications, and equivalents, which may be included within the spirit and scope of the various embodiments as defined by the appended claims. Furthermore, in this Detailed Description, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

Some portions of the detailed descriptions which follow are presented in terms of procedures, logic blocks, processing, and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities may take the form of electrical or magnetic signals capable of being stored, transferred, combined, compared, and otherwise manipulated in an electronic system, device, and/or component.

It should be borne in mind, however, that these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “determining,” “communicating,” “taking,” “comparing,” “monitoring,” “calibrating,” “estimating,” “initiating,” “providing,” “receiving,” “controlling,” “transmitting,” “isolating,” “generating,” “aligning,” “synchronizing,” “identifying,” “maintaining,” “displaying,” “switching,” or the like, refer to the actions and processes of an electronic item such as: a processor, a sensor processing unit (SPU), a processor of a sensor processing unit, an application processor of an electronic device/system, or the like, or a combination thereof. The item manipulates and transforms data represented as physical (electronic and/or magnetic) quantities within the registers and memories into other data similarly represented as physical quantities within memories or registers or other such information storage, transmission, processing, or display components.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles. In aspects, a vehicle may comprise an internal combustion engine system as disclosed herein.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example device vibration sensing system and/or electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration. One or more components of an SPU or electronic device described herein may be embodied in the form of one or more of a “chip,” a “package,” an Integrated Circuit (IC).

Hereinafter, embodiments will be described in detail with reference to the accompanying drawings, and the same or corresponding components are denoted as the same reference numeral regardless of the reference numerals, and overlapping descriptions thereof will be omitted.

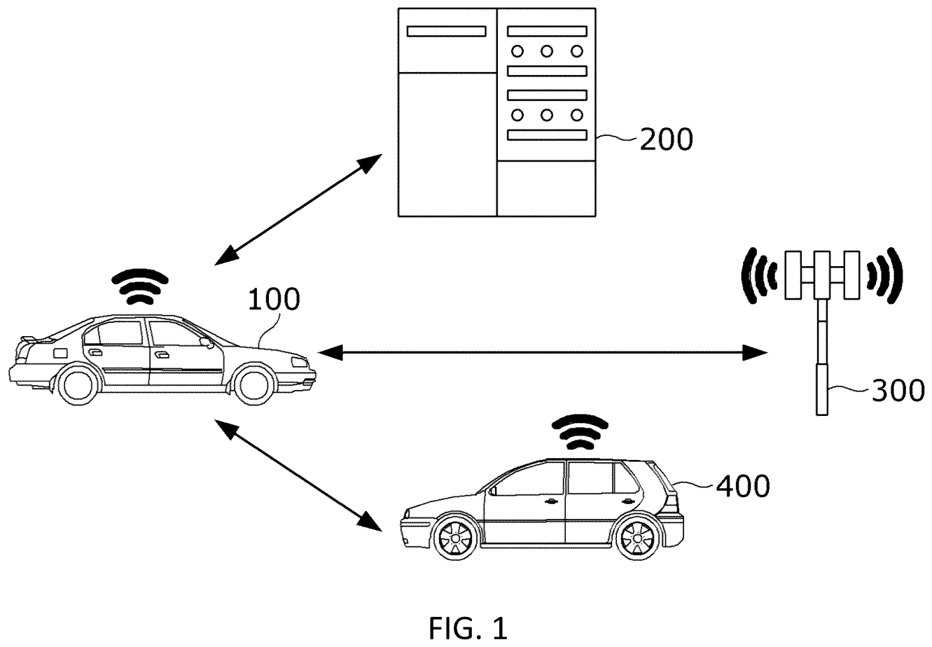

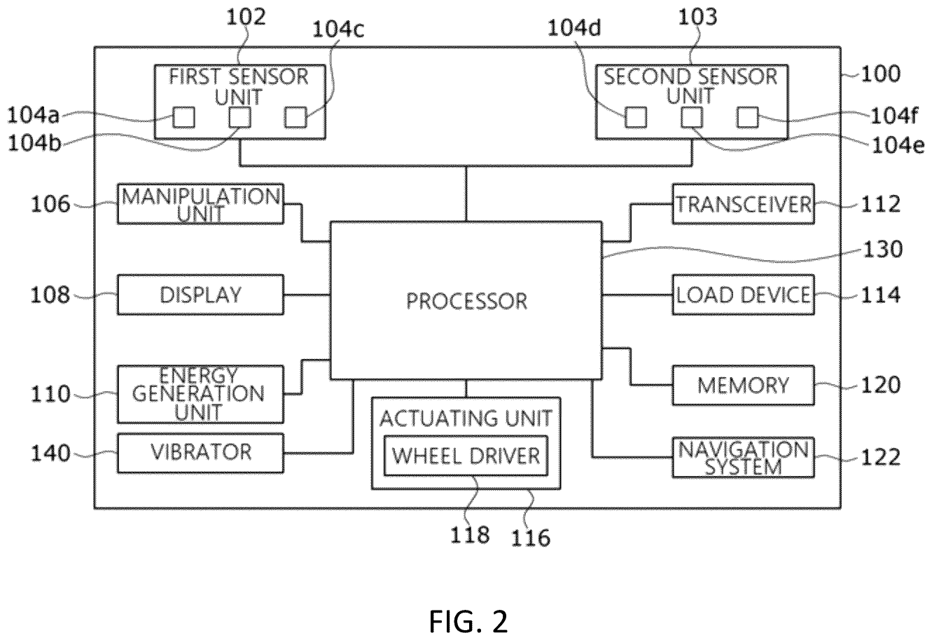

FIG. 1 is an exemplary view showing a vehicle 100 configured to communicate with another device 400 to transmit and receive data, and FIG. 2 is a view showing a module that constitutes a vehicle according to an exemplary embodiment of the present disclosure. A vehicle will be described with reference to FIGS. 1 and 2.

Referring now to FIG. 1, a vehicle 100 may be configured to be driven based on electric energy or fossil energy. In the case of electric energy, the vehicle 100 may be, for example, a pure battery-based vehicle driven by only a high-voltage battery or may adopt a gas-based fuel cell as an energy source. In addition, a fuel cell may use various types of gases that may generate electric energy, and the gas may be charged to the vehicle 100, for example, in a liquefied state. Here, the gas may be, for example, hydrogen. However, the present disclosure is not limited thereto, and various gases may be applied. In the case of fossil energy, the vehicle 100 may be driven based on fuel such as gasoline, diesel, liquefied gas, etc. and provided with an internal combustion engine that drives an actuating unit 116 by combustion of the fuel. The engine may be included in an energy generation unit 110 from the perspective of providing a driving rotational force of a wheel to a wheel driver 118. As another example, the vehicle 100 may be configured to drive the actuating unit 116 selectively using an internal combustion engine based on fossil energy and the energy of an electric battery and/or may be a hybrid-type vehicle.

The vehicle 100 may comprise a movable device. The vehicle 100 may comprise a ground vehicle configured to travel on the ground and may comprise a typical passenger or commercial vehicle, a purpose built vehicle (PBV), etc. The vehicle 100 may be a four-wheeled vehicle, for example, a passenger car, an SUV, or a small truck, or a vehicle with more than four wheels, for example, a bus, a large truck, a container transport vehicle, a heavy equipment vehicle, etc. Here, the ground vehicle may be referred to as not only a vehicle that moves on land but also a vehicle that moves underground. The vehicle 100 may be a robot in a broad sense, such as a means of transportation, and the robot may move using wheels, tracks, or other moving modules. In the present disclosure, a ground mobility device such as a ground vehicle is mainly described, but, unless it contradicts the present disclosure, the present embodiments may also be applied to air mobility devices, such as, e.g., an advanced air mobility (AAM), an aircraft, etc., and water mobility devices, such as, e.g., a ship, a submarine, etc.

The vehicle 100 may be configured to travel under autonomous driving control, and the autonomous driving may be implemented as semi-autonomous driving or full autonomous driving. The full autonomous driving may be provided as autonomous movement in which a processor 130 of the vehicle 100 has full control authority without user intervention even when a traveling situation is uncertain. The semi-autonomous driving may be provided as autonomous movement that requires driver intervention depending on a specific traveling situation. The semi-autonomous driving may be implemented by allowing a user to perform manual driving by deactivating autonomous driving in case of situation and transferring control authority to the user. According to the level of the autonomous driving defined by the Society of Automotive Engineers (SAE), the semi-autonomous driving may correspond to autonomous driving levels 1 to 4, and the full autonomous driving may correspond to autonomous driving level 5.

The vehicle 100 may be configured to communicate with one or more other devices 200 and 300 and/or another vehicle 400. The other devices may comprise, for example, a server 200 configured to support various control, state management, and traveling of the vehicle 100, an intelligent transportation system (ITS) device 300 configured to receive information from an ITS, various types of user devices, etc. The server 200 may comprise, for example, an external device operated by a vehicle manufacturer or provided to service autonomous driving and may be configured to receive connected data of the vehicle 100 or transmit data required for autonomous driving. To support autonomous driving and various services of the vehicle 100, the server 200 may be configured to transmit various types of information and/or software modules that may be used for controlling the vehicle 100 to the vehicle 100 in response to the request and data transmitted from the vehicle 100 and the user device.

The ITS device 300 may comprise, for example, a road side unit (RSU) and may be configured to exchange vehicle recognition data, traveling control and state data, surrounding environmental data of a vehicle, map data, etc. with the vehicle 100 through a vehicle-to-infrastructure (V2I) configured to provide assistance to a user driving his or her vehicle or support the autonomous driving of the vehicle 100. The vehicle 100 may be configured to exchange the above listed data with another vehicle 400 through a vehicle-to-vehicle (V2V) to support manual driving or autonomous driving.

The vehicle 100 may be configured to communicate with another vehicle and/or other devices based on cellular communication, wireless access in vehicular environment (WAVE) communication, dedicated short range communication (DSRC), short-range communication, or another communication method.

For example, the vehicle 100 may be configured to use a communication network such as, e.g., Long Term Evolution (LTE) or 5G, a Wi-Fi communication network, a WAVE communication network, etc. as a cellular communication network to communicate with the server 200, the ITS device 300, and another vehicle 400. As another example, the DSRC or the like used in the vehicle 100 may be used for communication between vehicles. A communication method between the vehicle 100, the server 200, the ITS device 300, another vehicle 400, and the user device is not limited to the above embodiment.

FIG. 2 illustrates a view showing a module that constitutes a vehicle according to an exemplary embodiment of the present disclosure.

The vehicle 100 may comprise a first sensor unit 102, a manipulation unit 106, a display 108, a load device 114, a transceiver 112, and/or other suitable components.

The first sensor unit 102 may comprise various types of detectors for detecting various states and situations that occur in an external environment, internal system, user manipulation, and boarding space of the vehicle 100.

Specifically, the first sensor unit 102 may comprise an outer-facing camera 104a, a lidar sensor 104b, a radar sensor 104c, etc., to recognize dynamic and static objects present outside the vehicle 100. The camera 104a may be configured to recognize an external object as an image while used in the vehicle 100 to generate image data and transmit the image data to the processor 130. The lidar sensor 104b may be configured to generate point cloud data as data of the recognized external object and transmit the point cloud data to the processor 130 in order to generate three-dimensional spatial information that identifies at least the shape of the external object. The radar sensor 104c may be configured to emit radio waves of a specific frequency to a peripheral area of the vehicle 100 to generate radar data through radio waves reflected from the external object in order to identify the presence, a relative distance, speed, direction, etc. of the external object. In the present disclosure, the lidar sensor 104b is provided as an example, but in another example, the lidar sensor 104b may not be mounted.

The first sensor unit 102 may be configured to generate object recognition information based on sensing data. The object recognition information may comprise information about whether an object is present, position information of the object, distance information between the vehicle 100 and the object, and relative speed information between the vehicle 100 and the object. In an embodiment, the external object may comprise various objects related to the driving of the vehicle 100.

A second sensor unit 103 may comprise a positioning sensor 104d, a wheel sensor 104e, an attitude sensor 104f, etc., to check a position, speed, driving attitude, etc., of the vehicle. The attitude sensor 104f may comprise a gyro sensor, an angular velocity sensor, an acceleration sensor, etc. The attitude sensor may comprise an inertial measurement unit (IMU) sensor and may comprise a 3-axis accelerometer and a 3-axis angular velocity meter. The attitude sensor may be configured to measure acceleration in a progress direction x of the vehicle 100, acceleration in a transverse direction y, acceleration in a height direction z, and yaw, pitch, and roll as an angular speed of the vehicle.

The second sensor unit 103 may be configured to generate vehicle traveling information based on the sensing data. The vehicle traveling information may comprise information generated based on data detected by various sensors installed in the vehicle. For example, the vehicle traveling information may comprise vehicle attitude information, vehicle speed information, vehicle tilt information, vehicle weight information, vehicle direction information, vehicle battery information, vehicle fuel information, vehicle tire pressure information, vehicle steering information, vehicle room temperature information, vehicle room humidity information, pedal position information, vehicle engine temperature information, etc.

In addition, the vehicle traveling information may comprise route information. The route information may comprise information generated based on a destination input by a vehicle user through the manipulation unit 106. The route information may comprise information in which a traveling route from a current position of a host vehicle to a destination is displayed on map information when the destination is set. When the destination is not set, the route information may comprise information that includes a road on which the vehicle is currently traveling and a future traveling route including the road.

A biometric information collector 105 may be provided on a head mounting device and a vibration seat and may collect biometric information of a passenger experiencing a virtual environment driving content.

The biometric information collector 105 may be provided on the vibration seat and may be configured to measure a heart rate of the passenger.

The biometric information collector 105 may be provided in a vibration seat 20. The biometric information collector 105 may comprise at least one of a photoplethysmography (PPG) sensor, an electrocardiography (ECG) sensor, and an acoustic sensor.

The PPG sensor may be formed of a light-emitting diode (LED) and a light sensor. The PPG sensor may be configured to measure the amount of blood using light. The PPG sensor may be configured to measure a heart rate through a principle in which light is emitted on skin from the LED and the amount of light reflected while passing through the skin and blood vessels varies depending on the amount of blood.

The ECG sensor may comprise one or more electrodes attached to the skin and a signal processing circuit. The ECG sensor may be configured to detect electrical activity that occurs when the heart beats through the electrodes attached to the skin.

The acoustic sensor may comprise a high-sensitivity microphone and a signal processing system. The acoustic sensor may be configured to record heartbeat sounds using a microphone and analyze the heartbeat sounds to measure a heart rate.

In addition, the biometric information collector 105 may be provided on a backrest and base cushion of a vehicle seat to measure a load of a passenger.

The manipulation unit 106 may be formed as a module manipulated by a user for driving. For example, the manipulation unit 106 may be a steering wheel for manual driving, an automatic or manual transmission, an accelerator pedal, a brake pedal, etc. The manipulation unit 106 may further comprise an interface configured for using, deactivating, and selecting a specific function of an autonomous driving mode requested by the user so that the user may use the autonomous driving function. To receive various requests related to autonomous driving, the manipulation unit 106 may comprise, for example, a hard type interface provided at a predetermined location in the vehicle 100 or a soft type interface that may be touched on the display 108. According to specifications of the autonomous driving vehicle, at least one of the steering wheel, the transmission, and the pedals may be omitted. As another example, the manipulation unit 106 may comprise a module that receives a control request of the user for the load device 114 in addition to driving control.

The display 108 may be configured to serve as a user interface. The display 108 may be configured to be controlled by the processor 130 to display an operation state, a control state, route/traffic information, and the remaining energy information of the vehicle 100, content requested by a driver, etc. In addition, the display 108 may be formed as a touch screen capable of detecting the input of the driver to receive the request of the driver that instructs the processor 130.

The load device 114 may be mounted on the vehicle 100 and may be a type of non-driving electric device not including a driving power system such as the wheel driver 118 and the like. The load device 114 may comprise an auxiliary device configured for receiving power from the energy generation unit 110 and may be, for example, various devices installed on an air conditioning system, a lighting system, a seat system, and the vehicle 100. In the present disclosure, a cooling/heating system for cooling or heating at least one of a battery, a fuel cell, an internal combustion engine, an air conditioning system, and a specific portion of the vehicle 100 may be further included.

The transceiver 112 may be configured to support mutual communication with the server 200, the ITS device 300, another vehicle 400, etc. The transceiver 112 may comprise, for example, a module for processing cellular communication, WAVE, DSRC communication, etc. In the present disclosure, the transceiver 112 may be configured to transmit data generated or stored during driving to the server 200 and receive a data and software module transmitted from the server 200. The transceiver 112 may be configured to support communication with an electronic device of a passenger in the vehicle 100. In the present disclosure, the vehicle 100 may be configured to transmit and receive data used in the method according to the present disclosure with an external device through the transceiver 112.

For example, the transceiver 112 may be configured to receive traffic signal information from a traffic signal controller and provide the traffic signal information to the processor 130. In addition, the transceiver 112 may be configured to receive a control signal information from the traffic signal controller and provide the control signal to the processor 130.

In addition, the vehicle 100 may comprise the energy generation unit 110 and the actuating unit 116.

The energy generation unit 110 may be configured to generate and supply power and electric power that are used in a driving power system and a non-driving power system, such as the actuating unit 116. The non-driving power system may comprise, for example, the first sensor unit 102, the manipulation unit 106, the display 108, the load device 114, the transceiver 112, etc., but is not limited thereto, and may comprise various components for implementing sensing, interface, communication, and convenience functions other than components directly involved in a driving operation. When the vehicle 100 is driven based on electric energy, the energy generation unit 110 may be provided as, for example, an electric battery charged from the outside or provided as a combination of an electric battery and a fuel cell that charges the battery. In the case of a combination of the electric battery and the fuel cell, the energy generation unit 110 may comprise a tank for storing a material used to produce power of the fuel cell, for example, liquefied hydrogen. When the vehicle 100 is driven based on fossil energy, the energy generation unit 110 may be formed of an internal combustion engine. In addition, when the vehicle 100 is a hybrid type, the energy generation unit 110 may be provided as a combination of the internal combustion engine and the electric battery.

The actuating unit 116 may comprise at least one module that implements a driving operation and perform at least one driving operation of longitudinal control such as acceleration and deceleration and transverse control such as steering according to a user request from the manipulation unit 106. To perform the driving operation according to the manual manipulation of the user or the instruction of the processor 130 according to autonomous driving, the actuating unit 116 may comprise the wheel driver 118, and a mechanical component and electronic module for implementing the driving operation of the wheel driver 118. When the vehicle 100 is operated based on electric energy, the vehicle 100 may comprise an assembly for transmitting the requested driving operation to the wheel driver 118. When the vehicle 100 is operated based on fossil energy, the actuating unit 116 may comprise a transmission and a gear module for transmitting the power of an internal combustion engine.

The wheel driver 118 may comprise a plurality of wheels, a driving force generation module for generating a driving force to impart the driving force to wheels or transmitting the driving force, a brake module for decelerating the driving of the wheels, a steering module for achieving transverse control of the wheels, etc. When the vehicle 100 is driven based on electric energy, the driving force generation module may be provided as a motor assembly for generating a driving force based on power output from the electric battery. The brake module of the electricity-based vehicle 100 may further have a regenerative brake function.

A navigation system 122 may be configured to provide navigation information. The navigation information may comprise at least one of map information, set destination information, route information according to destination setting, information about various objects on a route, lane information, and current position information of the vehicle.

The navigation system 122 may be configured to receive information from an external device through the transceiver 112 and update previously stored information. According to an embodiment, the navigation system 122 may be classified as a subcomponent of the manipulation unit 106.

In addition, the vehicle 100 may comprise a memory 120 and the processor 130.

The memory 120 may be configured to store applications and various types of data for controlling the vehicle 100 and load the applications or read or write the data at the request of the processor 130.

The processor 130 may be configured to perform the overall control of the vehicle 100. The processor 130 may be configured to execute applications and instructions that are stored in the memory 120.

In an embodiment, the components may have different functions and capabilities in addition to the above description and include additional components in addition to those to be described below. In addition, in one embodiment, each component may be implemented using one or more physically separated devices, or implemented by one or more processors 130 or a combination of the one or more processors 130 and software, and may not be clearly distinguished in specific operations unlike the shown example.

The memory 120 may comprise a database (DB). In addition, the memory 120 may comprise a non-transitory storage medium for storing instructions executed by the processor 130. The memory 120 may comprise at least one of a random access memory (RAM), a static RAM (SRAM), a read only memory (ROM), a programmable ROM (PROM), an electrically erasable and programmable ROM (EEPROM), an erasable and programmable ROM (EPROM), a hard disk drive (HDD), a solid state disk (SSD), an embedded multimedia card (eMMC), a universal flash storage (UFS), and/or a web storage.

The processor 130 may comprise at least one of processing devices such as an application specific integrated circuit (ASIC), a digital signal processor (DSP), a programmable logic device (PLD), a field programmable gate arrays (FPGA), a central processing unit (CPU), a microcontroller, and/or a microprocessor.

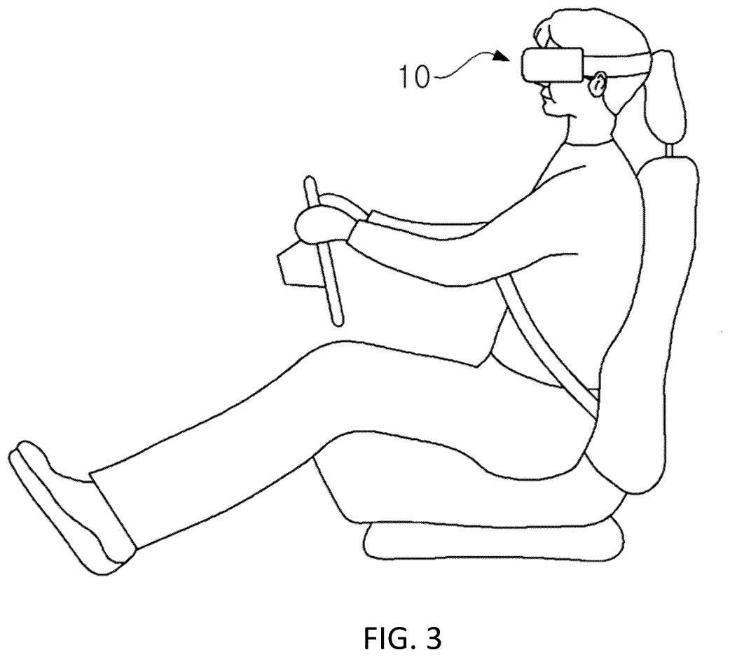

FIG. 3 is a view for describing an environment for providing a virtual environment driving content according to an embodiment. Referring to FIG. 3, virtual content according to the embodiment may be applied to a user of an autonomous driving vehicle who experiences a virtual environment content while wearing a head-mounted display (HMD) in a metaverse environment or the like.

In an embodiment, an HMD 10 may comprise a display device disposed in a vehicle and worn on a head of a user. The HMD 10 may be configured to be mainly used in virtual reality (VR) and augmented reality (AR) applications and may be configured to provide an immersive visual experience through a display disposed in front of eyes of the user.

The HMD 10 may comprise a display panel that provides images to the user using two small screens or one large screen, and a lens that is positioned between the display and the eyes of the user and adjusts the image to the eyes.

The HMD 10 may be configured to track head movement of the user using a head tracking technology implemented through a gyroscope, an accelerometer, a magnetic field sensor, etc. and adjust a field of view of a screen.

The HMD 10 may be configured to provide a virtual environment driving content to the user wearing it. In an embodiment, the virtual environment driving content may include virtual images and virtual sounds generated based on the driving environment of a vehicle driver.

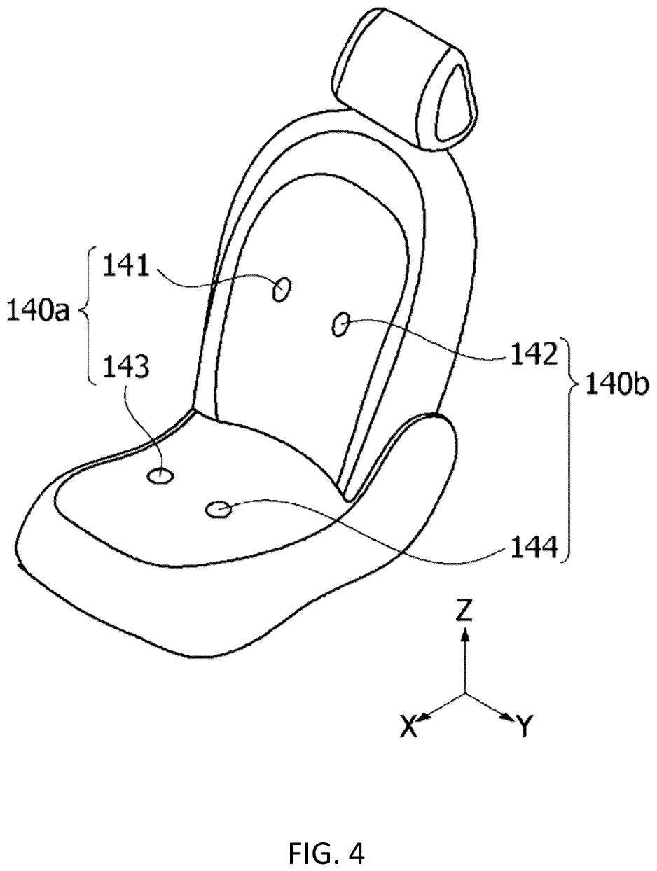

FIG. 4 is a view for describing an operation of a vibrator according to the embodiment. Referring to FIG. 4 together, vibrators 140 may be provided on a backrest and base cushion of the seat and may independently output a vibration signal. The vibrator 140 may be disposed to be embedded in an empty space of the backrest and the base cushion of the seat, in which two vibrators 141 and 142 may be disposed to be spaced a predetermined distance from each other on the backrest and two vibrators 143 and 144 may be disposed to be spaced a predetermined distance from each other on the base cushion. The vibrators 141 to 144 may be configured to operate independently under the control of the processor 130 and output predetermined vibration signals.

Each vibrator 140 may comprise a frame forming an exterior, a voice coil that is installed in the frame and generates a magnetic field when an electrical signal is applied, at least one magnet that interacts with the voice coil by the magnetic field of the voice coil and vibrates at a predetermined frequency, a vibrator that transmits the vibration of the magnet to the human body, etc. When an electrical signal is applied to the voice coil of the vibrator 140 under the control of the processor, a magnetic field proportional to an intensity of the electrical signal is generated by the voice coil, and when such a magnetic field interacts with the magnet, the magnet vertically vibrates at the predetermined frequency. When such a vibration signal is output through the vibrator and transmitted to the human body, the human body recognizes a predetermined acoustic signal.

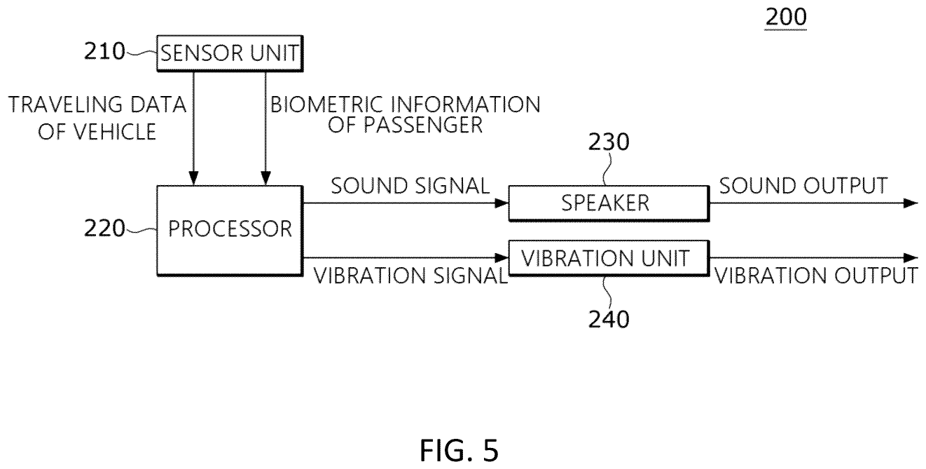

FIG. 5 is a view for describing an operation of the vehicle according to the embodiment.

Referring to FIG. 5, a sensor unit 210 may be configured to detect traveling data of the vehicle and biometric information of the vehicle passenger. The sensor unit 210 of FIG. 5 may be a component including the second sensor unit 103 and the biometric information collector of FIG. 2.

Biometric information may comprise at least one of heart rate information, respiration information, skin stimulation information, and movement information of the passenger.

Regenerative braking information may comprise regenerative braking stage information.

The sensor unit 210 may comprise the IMU sensor that collects traveling data of the vehicle. The IMU sensor may be composed of a three-axis accelerometer, a three-axis gyroscope, and a magnetometer and may be configured to detect the movement of the vehicle. The three-axis accelerometer may be configured to measure changes in acceleration in a front-rear direction x, a left-right direction y, and a vertical direction z of the vehicle and analyze dynamic changes during deceleration. In particular, the IMU sensor may be configured to measure deceleration during the operation of regenerative braking and detect the suddenness of a change in speed. In addition, the 3-axis gyroscope may be configured to measure rotation (Roll, Pitch, Yaw) information of the vehicle and detect a sudden change in direction during deceleration. In addition, the magnetometer may be configured to serve to correct a direction and movement of the vehicle and provide more accurate data. Accordingly, a deceleration pattern of the vehicle can be accurately identified, and the possibility of motion sickness can be evaluated.

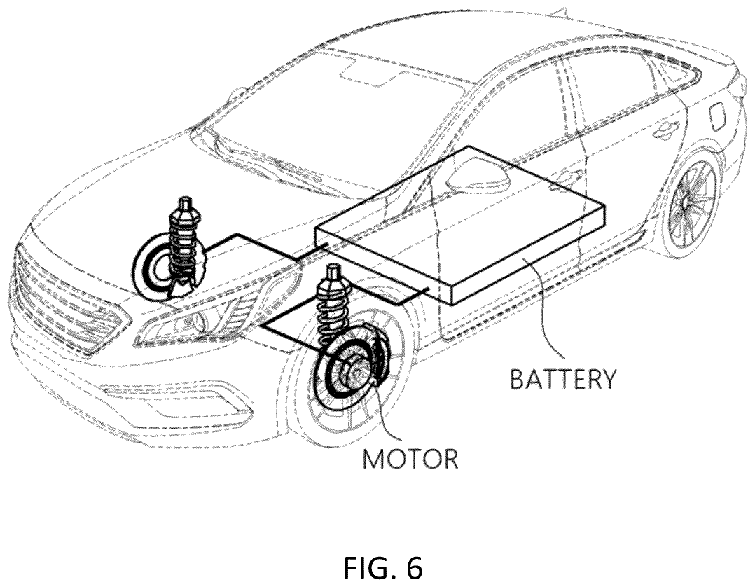

In addition, embodiments of the present disclosure may be configured to provide feedback for reducing motion sickness in connection with a regenerative braking control system. Referring to FIG. 6 together, the regenerative braking control system may be configured to adjust the intensity of deceleration step by step and is generally set as 3 or 5 stages in electric vehicles. When the regenerative braking of the vehicle is strongly performed, the possibility of motion sickness is increased by sudden deceleration, and thus it is important to detect this moment and provide prompt feedback. For example, synchronized vibrations may be provided to the seat at a moment when the intensity of the regenerative braking is high to induce sensory correction, or soft background sounds may be provided when the deceleration changes irregularly to mitigate the discomfort of the passenger. In addition, by analyzing the regenerative braking pattern based on machine learning, when a specific deceleration pattern causes motion sickness, it is possible to predict it in advance and appropriately respond thereto.

The biometric information of the passenger acts as an important factor in detecting whether motion sickness occurs. To this end, the sensor unit 210 may be configured to monitor physiological changes in real time using heart rate sensors (PPG and ECG), a respiration sensor, and a galvanic skin response (GSR) sensor. The heart rate sensor evaluates the possibility of motion sickness using a change in heart rate that increases when the sympathetic nerve is activated when motion sickness occurs. The PPG sensor may be attached to a wrist, finger, ear, etc. to detect a change in blood flow, and the ECG sensor may be configured to detect an increase in a heart rate due to the sympathetic nerve activated when motion sickness occurs through more precise heart rate rhythm analysis.

The respiration sensor uses characteristics that a respiration pattern changes when motion sickness occurs. Generally, since short and rapid respiration occurs when motion sickness occurs, it may be detected to determine the possibility of motion sickness. The respiration sensor may be configured to be worn on the chest in the form of a belt or implemented as a built-in vehicle seat and may be configured to compare a normal respiration pattern with a respiration pattern when motion sickness occurs and determine whether there is an abnormality.

The GSR sensor detects the anxiety and stress response of the passenger and evaluates the possibility of motion sickness. The GSR sensor measures a change in minute electrical conduction of the skin on the palm or fingers, and a skin conductance increases when the palm sweat increases due to the activation of the sympathetic nerve. Using this, stress and anxiety may be measured, and prompt feedback may be provided when the skin conductance exceeds a predetermined threshold value.

In addition, the sensor unit 210 may comprise a room camera that captures an image of an interior of the vehicle. The room camera may be configured to capture an image of the interior of the vehicle including movement information of a vehicle occupant and generate image data.

A processor 220 may be configured to determine a relationship between the regenerative braking information of the traveling data of the vehicle, the acceleration information generated during regenerative braking, and the biometric information, analyze the risk of motion sickness of the passenger based on the correlation, and generate and output a motion sickness reduction feedback signal of at least one of a sound signal and a vibration signal according to the risk of motion sickness. The processor 220 of FIG. 5 may be the same component as the processor 130 of FIG. 2.

The processor 220 may be configured to analyze the correlation between the regenerative braking information of the traveling data of the vehicle, the acceleration information generated during regenerative braking, and the biometric information and analyze the risk of motion sickness of the passenger based on the correlation.

The processor 220 may be configured to analyze the correlation between the regenerative braking information, the pre-acceleration information, and the biometric information and analyze the risk of motion sickness.

A motion sickness prediction model may be configured to learn a change in the biometric information according to a regenerative braking stage included in the regenerative braking information and acceleration information.

The processor 220 may be configured to determine a relationship between a vehicle driving environment and a biological response of a passenger by applying machine learning techniques, and in particular, predict the risk of motion sickness using an algorithm based on a transformer model.

The regenerative braking of a vehicle is a process of transforming kinetic energy into electrical energy of the vehicle when decelerating during traveling, and at this time, various levels of changes in acceleration may occur depending on the deceleration pattern of the vehicle. The change in acceleration directly affects the body of the passenger, and under a specific condition, the possibility of causing motion sickness increases.

The processor 220 may be configured to analyze a regenerative braking stage and acceleration information accordingly, and at the same time, evaluate correlation with biometric information of the passenger and predict the risk of motion sickness.

The motion sickness prediction model may be configured to learn the change in biometric information according to the regenerative braking stage and the change in acceleration of the vehicle. As described above, the biometric information may comprise physiological signals, such as a heart rate, GSR, skin temperature, respiration pattern, etc. of a passenger, and these signals are closely related to whether a user experiences motion sickness. The motion sickness prediction model may be configured to analyze changes in these pieces of the biometric information and based on this, may be configured to determine the risk of motion sickness of the passenger.

The motion sickness prediction model performs motion sickness prediction using an encoder-decoder structure of the transformer model, and thus can effectively map the regenerative braking stage of the vehicle and the biometric information of the passenger to predict sensory conflict.

The motion sickness prediction model may be composed of an encoder and a decoder. The encoder may be configured to receive the regenerative braking information of the vehicle and the biometric information of passenger and transform them into a feature space, and based on this, the decoder may be configured to output the motion sickness risk. In particular, the encoder may be configured to learn the biometric information of the passenger to generate sensory conflict, and the decoder may be configured to derive motion sickness prediction results based on the sensory conflict.

In addition, the motion sickness prediction model can increase the accuracy of motion sickness prediction by applying an attention mechanism in the encoder-decoder structure. The attention mechanism is a method that weights important parts of input data, and thus can effectively extract factors that have a greater influence on motion sickness among the regenerative braking information of the vehicle. For example, when a specific regenerative braking pattern has a significant influence on the biometric response of the passenger, the attention mechanism may be configured to increase the weight for the corresponding pattern so that the motion sickness prediction model may reflect it more accurately.

In addition, the processor 220 may be configured to evaluate a cyber motion sickness stage and user sensitivity by comparing the motion sickness prediction model with the user sensory conflict and analyzing the result of the comparison. The cyber motion sickness is motion sickness that occurs when using a VR environment or an in-vehicle digital interface and may be caused by factors different from those of general vehicle motion sickness. The processor 220 may be configured to analyze user sensory conflict data to identify the stage of the cyber motion sickness and user-specific sensitivity, and thus provide a motion sickness reduction solution tailored to each user.

The processor 220 may be configured to generate and output a motion sickness reduction feedback signal including a sound signal depending on the risk of motion sickness.

The processor 220 may be configured to generate and output a motion sickness-reduction feedback signal for each step based on the risk of motion sickness.

When the acceleration generated during regenerative braking is within a first threshold range, the processor 220 may be configured to generate a sound signal for stabilizing a heart rate and output the generated sound signal to the speaker 230 in the vehicle.

When the acceleration generated during regenerative braking is within a second threshold range, the processor 220 may be configured to generate and output a sound signal for stabilizing a respiration pattern.

The processor 220 may be configured to control the physiological response of the passenger and alleviate motion sickness by generating a sound signal corresponding to the change in acceleration generated during the regenerative braking process of the vehicle.

The processor 220 may be configured to generate the sound signal for stabilizing a heart rate when the acceleration generated during the regenerative braking process is within the first threshold range (0.1 g to 0.29 g) and generate the sound signal for stabilizing a respiration pattern and output the generated sound signal to the speaker in the vehicle when the acceleration is within the second threshold range (0.3 g or more).

The processor 220 may be configured to provide a stage-specific music content based on the acceleration data according to the regenerative braking by applying a data integration and transformation function. That is, the processor 220 may be configured to generate a sound signal using a piano performance technique connected to a heart rate when the change in acceleration is relatively small (0.1 g to 0.29 g) and stabilization of the heart rate is required and generate a sound signal using a cello performance technique that induces long respiration when the change in acceleration is relatively large (0.3 g or more) and stabilization of the respiration pattern is required.

Heart rate synchronization is based on the physiological principle of synchronizing a biorhythm of a human body with external stimulus and is a technology in which a sound signal with a specific rhythm or frequency affects and controls rhythms of human body organs. In the embodiment, the goal is to induce a heart rate to about 65 bpm within the normal range (60 to 100 bpm), and to this end, the processor 220 may be configured to use piano performance. The piano performance applies the Ab Major key that provides a sense of stability in connection with a heart rate of a user and makes an auditory comfortable environment using less complex diatonic harmony. Ab Major is a key that conveys the feeling of “andante” and may provide the effect of gradually decreasing a heart rate. In addition, diatonic harmony contributes to implementing the feeling of a natural heartbeat so that a user can maintain a more stable heartbeat pattern.

Meanwhile, a long-respiration-connected cello performance technique is a method of providing psychological stability by maintaining a respiration of a user for a long time, and the processor 220 may be configured to musically transform the long-respiration method used in meditation techniques and apply the transformed long-respiration method. In the embodiment, the processor 220 may be configured to use a legato technique of cello performance. Legato is a performance method that smoothly connects notes without breaking them and may naturally induce the long respiration of a listener. In the embodiment, the processor 220 may be configured to use the cello performance technique to maintain a long and soft melody in connection with the respiration pattern of the user and provide a constant rhythm and soft tone by minimizing position movement when producing high-pitched sounds.

The processor 220 may be configured to analyze the risk of motion sickness, generate a motion sickness reduction feedback signal for each step, and output an appropriate sound signal according to a progress degree of motion sickness. For example, the processor 220 may be configured to preferentially apply piano performance that adjusts a heart rate in an initial motion sickness stage and additionally provide cello performance that stabilizes respiration when motion sickness is severe. Accordingly, the processor 220 may be configured to provide a motion sickness reduction solution optimized for a biological response of an individual user.

The processor 220 may be configured to generate a motion sickness reduction feedback signal including a vibration signal according to the risk of motion sickness and output the generated motion sickness reduction feedback signal including a vibration signal through a vibration unit 240 in the vehicle.

The processor 220 may be configured to adjust an intensity and pattern of the vibration signal according to the risk of motion sickness.

The processor 220 may be configured to adjust the intensity and pattern of the vibration signal in consideration of the biological response of the passenger and the risk of motion sickness and provide a more effective motion sickness reduction function by applying a sound-based haptic solution.

Motion sickness occurring during vehicle movement is caused by sensory conflict between vision, vestibular organ (balance sense of the ear), and somatosensory (detection of body movement), and the processor 220 may be configured to generate a vibration signal that minimizes the sensory conflict.

The processor 220 may be configured to evaluate the risk of motion sickness in real time, and thus may be configured to generate and output the motion sickness reduction feedback signal including the vibration signal. In addition, the processor 220 may be configured to perform optimized response for individual users by adjusting the intensity and pattern of the vibration signal according to the degree of motion sickness.

The processor 220 may be configured to provide haptic and vibration solutions connected to sounds according to the risk of motion sickness of the passenger using a dynamic content generation function. To this end, the vehicle according to the embodiment may be provided with an air pressure haptic technology based on an ergo motion seat and the vibration unit 240 based on a vibro music seat.

The ergo motion seat may be configured to serve to correct a seating posture of a rear seat passenger and alleviate motion sickness by adjusting air pressure using seven air pockets. Haptic feedback using air pressure can help prevent motion sickness by automatically correcting the posture of the passenger according to the movement of the vehicle and is particularly effective in reducing discomfort caused by an unnatural posture during long-distance travel. In addition, the ergo motion seat may be configured to provide a massage function using the air pocket located on a backrest portion, thereby relieving muscle tension that may occur due to motion sickness and inducing relaxation of the passenger.

The vibro music seat may be configured to generate vibrations using 4 to 8 actuators, thereby minimizing sensory conflict and enhancing VR content-based immersion. The processor 220 may be configured to provide vibrotactile stimulation using a method of minimizing mismatch between an image and body movement to reduce sensory conflict. For example, when the user looks forward during vehicle deceleration, the processor 220 may be configured to visually feel the vehicle that seems to be stopping, but the vestibular organ may still detect the decelerating movement. Such mismatch may cause motion sickness, and the processor 220 can decrease mismatch between pieces of sensory information by providing vibrations of a specific frequency according to a deceleration pattern of the vehicle in such a situation.

In addition, the vibro music seat may be configured to provide customized vibrations that enhance immersion in connection with a VR content. In a VR environment, mismatch between movement of a user and visual information may cause motion sickness, and the processor 220 generates a vibration signal that matches the movement of the VR content in such an environment so that the passenger can be more naturally immersed. For example, when an acceleration situation occurs in the VR content, the processor 220 may be configured to increase the intensity of vibration of the seat to imitate a sense of acceleration of an actual vehicle.

The processor 220 may be configured to analyze the risk of motion sickness and provide optimal haptic feedback according to a degree of motion sickness. In an initial stage of motion sickness, the processor 220 may be configured to induce posture correction and muscle relaxation using the ergo motion seat, and when motion sickness is severe, the processor 220 may be configured to provide a vibration signal that alleviates sensory conflict through the vibro music seat. Accordingly, the passenger can maintain a more comfortable state while the vehicle is moving, and discomfort caused by motion sickness can be minimized.

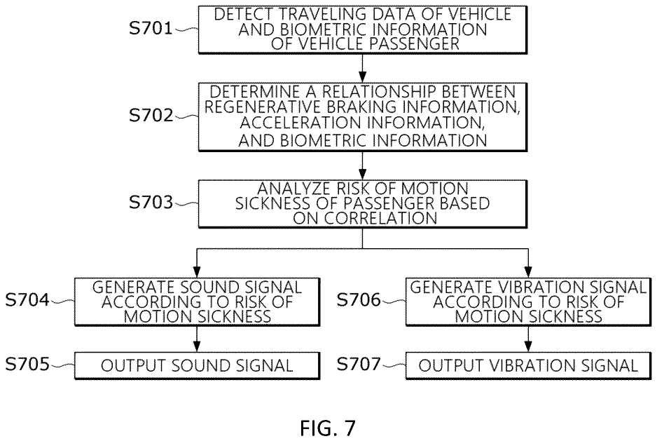

FIG. 7 is a flowchart showing a method of controlling a vehicle according to an embodiment. Referring to FIG. 7, the sensor unit detects traveling data of the vehicle and biometric information of the vehicle passenger (S701).

The processor analyzes correlation between the regenerative braking information of the traveling data of the vehicle, the acceleration information generated during regenerative braking, and the biometric information (S702).

The processor analyzes the risk of motion sickness of the passenger based on the correlation.

That is, the processor may be configured to analyze the risk of motion sickness by analyzing the correlation between the regenerative braking information, the acceleration information, and the biometric information using the motion sickness prediction model. The motion sickness prediction model may be configured to analyze the risk of motion sickness of each passenger by learning a change in biometric information according to the regenerative braking stage included in the regenerative braking information and the acceleration information (S703).

The processor generates a sound signal according to the risk of motion sickness (S704).

The processor outputs the generated sound signal through a speaker in the vehicle (S705).

In addition, the processor generates a vibration signal according to the risk of motion sickness (S706).

The processor outputs the vibration signal generated through the vibration unit of the vehicle seat (S707).

The term “unit” used in the present embodiment means a software or hardware component such as an FPGA or an ASIC, and the “unit” performs certain roles. However, the “unit” is not limited to software or hardware. The “unit” may be disposed in an addressable storage medium and configured to reproduce one or more processors. Accordingly, as an example, the “unit” includes components such as software components, object-oriented software components, class components, and task components, processes, functions, attributes, procedures, subroutines, segments of program code, drivers, firmware, microcode, circuits, data, database, data structures, tables, arrays, and variables. Functions provided in the components and “units” may be combined into a smaller number of components and “units” or separated into additional components and “units.” Additionally, the components and “units” may be implemented to reproduce one or more CPUs in a device or a security multimedia card.

According to a vehicle and a method of controlling a vehicle according to embodiments, an impact of regenerative braking during traveling of an electric vehicle can be analyzed, thereby alleviating motion sickness of passengers based on the analyzed impact.

Accordingly, it is possible to predict situations with a high possibility of motion sickness in advance and provide motion sickness reduction feedback based on sound and vibration to alleviate motion sickness.

In addition, it is possible to improve the riding comfort of electric vehicle passengers by providing a motion sickness reduction solution.

In addition, it is possible to alleviate motion sickness and make a comfortable environment in a vehicle.

In addition, by applying the motion sickness reduction solution, a regenerative braking function of the electric vehicle can be utilized more actively, thereby maximizing the energy efficiency of the vehicle.

Although the present disclosure has been described above with reference to exemplary embodiments, those skilled in the art will understand that the present disclosure may be modified and changed variously without departing from the spirit and scope of the present disclosure as described in the appended claims.

What is claimed is:

1. A vehicle comprising:
a sensor unit;
one or more processors; and
a memory configured to store one or more programs executed by the one or more processors,
wherein:
the sensor unit is configured to detect:
traveling data of a vehicle; and
biometric information of a passenger of the vehicle, and
the processor is configured to:
determine a relationship between regenerative braking information of the traveling data of the vehicle, acceleration information generated during regenerative braking, and the biometric information;
determine a risk of motion sickness of the passenger based on the correlation; and
generate and output a motion sickness reduction feedback signal according to the risk of motion sickness.

2. The vehicle of claim 1, wherein the biometric information comprises at least one of heart rate information, respiration information, skin stimulation information, and movement information of the passenger.

3. The vehicle of claim 1, wherein the regenerative braking information comprises regenerative braking stage information.

4. The vehicle of claim 1, wherein the processor comprises a motion sickness prediction model configured to analyze the risk of motion sickness by analyzing the correlation between the regenerative braking information, the acceleration information, and the biometric information.

5. The vehicle of claim 4, wherein the motion sickness prediction model is configured to:
learn a change in the biometric information according to a regenerative braking stage included in the regenerative braking information and the acceleration information; and
predict the risk of motion sickness based on the change in the biometric information.

6. The vehicle of claim 1, wherein the motion sickness reduction feedback signal comprises at least one of a sound signal and a vibration signal.

7. The vehicle of claim 1, wherein the processor is configured to generate and output a motion sickness-reduction feedback signal for each step based on the risk of motion sickness.

8. The vehicle of claim 7, wherein the processor is configured to generate and output a sound signal for stabilizing a heart rate based on an acceleration generated during the regenerative braking being within a first threshold range.

9. The vehicle of claim 7, wherein the processor is configured to generate and output a sound signal for stabilizing a respiration pattern based on an acceleration generated during the regenerative braking being within a second threshold range.

10. The vehicle of claim 1, wherein the processor is configured to adjust an intensity and pattern of the vibration signal according to the risk of motion sickness.

11. A method of controlling a vehicle, which is performed by a computing device comprising a sensor unit, one or more processors, and a memory configured to store one or more programs executed by the one or more processors, the method comprising:
detecting, by the sensor unit, traveling data of a vehicle and biometric information of a passenger of the vehicle;
analyzing, by the processor, a correlation between regenerative braking information of the traveling data of the vehicle, acceleration information generated during regenerative braking, and the biometric information;
analyzing, by the processor, a risk of motion sickness of the passenger based on the correlation; and
generating and outputting, by the processor, a motion sickness reduction feedback signal according to the risk of motion sickness.

12. The method of claim 11, wherein the biometric information comprises at least one of heart rate information, respiration information, skin stimulation information, and movement information of the passenger.

13. The method of claim 11, wherein the regenerative braking information comprises regenerative braking stage information.

14. The method of claim 11, wherein the processor comprises a motion sickness prediction model that analyzes the risk of motion sickness by analyzing the correlation between the regenerative braking information, the acceleration information, and the biometric information.

15. The method of claim 14, wherein:
the analyzing of the correlation comprises learning, by the motion sickness prediction model, a change in the biometric information according to a regenerative braking stage included in the regenerative braking information and the acceleration information, and
the analyzing of the risk of motion sickness comprises predicting, by the motion sickness prediction model, the risk of motion sickness based on the change in the biometric information.

16. The method of claim 11, wherein the motion sickness reduction feedback signal comprises at least one of a sound signal and a vibration signal.

17. The method of claim 11, wherein the generating and outputting of the feedback signal comprises generating and outputting a motion sickness-reduction feedback signal for each step based on the risk of motion sickness.

18. The method of claim 17, wherein the generating and outputting of the feedback signal comprises generating and outputting a sound signal for stabilizing a heart rate based on an acceleration generated during the regenerative braking being within a first threshold range.

19. The method of claim 17, wherein the generating and outputting of the feedback signal comprises generating and outputting a sound signal for stabilizing a respiration pattern based on an acceleration generated during the regenerative braking being within a second threshold range.

20. The method of claim 11, wherein the generating and outputting of the feedback signal comprises adjusting an intensity and pattern of the vibration signal according to the risk of motion sickness.
